(19) United States
(12) Patent Application Publication
(10) Pub. No.: US 2006/0100533 A1
(43) Pub. Date: May 11, 2006

(54) Apparatus and method for measuring bio signals

(75) Inventors: Wan-taek Han, Hwaseong-si (KR); Hyung-sok Yeo, Yongin-si (KR); Ji-hyun Choi, Seoul (KR); Jin-sang Hwang, Suwon-si (KR); Jeong-whan Lee, Suwon-si (KR)

Correspondence Address: SUGHRUE MION, PLLC, 2100 PENNSYLVANIA AVENUE, N.W., SUITE 800, WASHINGTON, DC, 20037, US

(73) Assignee: SAMSUNG ELECTRONICS CO., LTD.

(21) Appl. No.: 11/267,291

(22) Filed: Nov. 7, 2005

(30) Foreign Application Priority Data
Nov. 6, 2004 (KR) 10-2004-0090129

(51) Int. Cl. A61B 5/02; A61B 5/04

(52) U.S. Cl. 600/508; 128/903; 600/509; 600/546; 600/374

(57) ABSTRACT

An apparatus and a method for measuring bio signals are provided. The apparatus includes a body, a plurality of electrodes, and a controller. The body is filled with predetermined packing material. The electrodes are positioned at an outermost portion of the body and touch the skin of a subject to measure bio signals from the subject. The controller is positioned inside of the body and connected with the electrodes to analyze the bio signals measured from the electrodes and output bio information of the subject.

### CROSS-REFERENCE TO RELATED PATENT APPLICATION

This application claims the benefit of Korean Patent Application No. 10-2004-0090129, filed on Nov. 6, 2004, in the Korean Intellectual Property Office, the disclosure of which is incorporated herein in its entirety by reference.

### BACKGROUND OF THE INVENTION

1. Field of the Invention

The present invention relates to an apparatus and a method for measuring bio signals, and more particularly, to an apparatus for measuring bio signals having a plurality of electrodes and a body in an integral type for measurement of bio signals and an associated method.

2. Description of the Related Art

As interest in personal health is increasing, home-based health care services where a user need not visit a professional institution such as a hospital are becoming more desirable. Much effort has been expended in developing a bio-signal detector whose use is not limited by space or time Particularly, as the number of people dieing of heart disease increases and the population rapidly ages, a continuously monitored electrocardiogram is becoming more necessary.

Electrocardiogram detection has generally been performed by experienced personnel under a controlled measurement environment. There are many limitations when an inexperienced user detects an electrocardiogram under everyday living conditions. That is, the inexperienced user is subject to obtaining inaccurate signals due to environmental conditions. The inaccurate signals may contain much noise and severe distortions. To meet the above-described necessities, a conventional apparatus with which a user can monitor bio signals during normal living conditions is under development. However, the apparatus for detecting the bio signals is inconvenient to use and is uncomfortable, since a gell-type contact electrolyte should be injected between electrodes and the user's skin. Further, the apparatus for detecting large signals has disadvantages in that not only is the sticking force of a sensor degraded due to biological changes of the user's body such as perspiration, but also reliability or accuracy of the obtained bio signals declines due to the inaccuracy of a sticking position since a user attaches the electrodes himself.

An apparatus for detecting bio signals is discussed in U.S. Pat. No. 6,286,899 and PCT publication No. 2002-0089667, which are incorporated herein by reference.

### SUMMARY OF THE INVENTION

The present invention provides an apparatus and a method for processing bio signals measured by a plurality of electrodes to detect bio information such as a heart rate.

According to an aspect of the present invention, there is provided an apparatus for detecting bio signals, which includes: a body filled with predetermined packing material; a plurality of electrodes positioned at an outermost portion of the body and in contact with a subject's skin, for measuring bio signals from the subject; and a controller positioned inside of the body and connected with the electrodes, for analyzing the bio signals measured by the electrodes to output bio information of the subject.

According to another aspect of the present invention, there is provided a method for detecting bio signals, which includes: attaching a body having a plurality of electrodes and filled with predetermined packing material to a subject; measuring the bio signals using the plurality of electrodes and selecting two bio signals among the bio signals; and analyzing the two bio signals to generate bio information.

### BRIEF DESCRIPTION OF THE DRAWINGS

The above and other features and advantages of the present invention will become more apparent by describing in detail exemplary embodiments thereof with reference to the attached drawings in which:

FIG. 1 illustrates a configuration of an apparatus for detecting bio signals according to the present invention;

FIG. 2 is a detailed block diagram of a controller of FIG. 1;

FIG. 3 illustrates two exemplarily signals selected among bio signals generated from electrodes shown in FIG. 1; and

FIG. 4 is a block diagram of an internal configuration of a mobile terminal shown in FIG. 1.

### DETAILED DESCRIPTION OF THE INVENTION

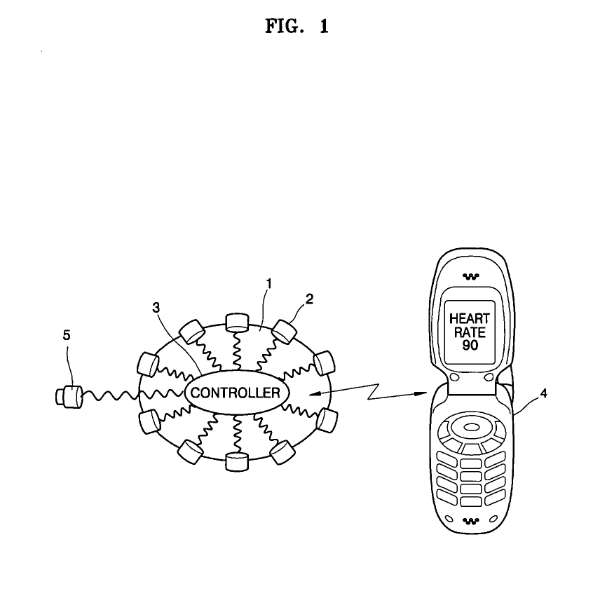

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. FIG. 1 illustrates a configuration of an apparatus for detecting bio signals according to the present invention. Referring to FIG. 1, the apparatus for detecting the bio signals includes a body 1, a plurality of electrodes 2, and a controller 3. A reference numeral 4 represents a mobile terminal 4 for receiving bio information detected and transmitted from the controller 3 and displaying the information or informing a user of the information by sound, such as, for example, a voice. Here, the mobile terminal 4 may be a terminal that can be carried by a user and can communicate with the controller 3. For example, the mobile terminal can be a separate apparatus for receiving and displaying the bio information outputted from the controller 3, or a related art communication terminal such as a personal digital assistant (PDA) or a cellular phone.

A reference numeral 5 is an input unit optionally provided, for requesting a user to measure the bio signals. The input unit is connected with the controller 3 and can be provided in the form of a button.

The body 1 is of a pack shape and has the controller 3 in its interior and the plurality of electrodes 2 at its outermost side. An inside of the pack is filled with elastic material such as, for example, liquid silicon, air, or a sponge and may be flexible so as to be attached anywhere on the body of a subject.

The electrodes 2 are connected with the controller 3 through a conductive wire made of flexible material so that they may easily make contact with the skin. The electrodes 2 may be manufactured of material such as, for example, metal or conductive plastics. The electrodes 2 contact and are electrically connected with the skin of the subject so as to electrically measure the bio signals and transmit the measured signals. Here, the respective electrodes may be preferably, but not necessarily, covered with hydrogen adhesive which is a conductive polymer adhesive. The electrodes are not limited to being covered with the hydrogen adhesive but a variety of material and structures known in the art may be used. Ideally, the bio signals may be measured by all of the electrodes 2 but some bio signals may not be measured by some of the electrodes depending on the three-dimensional structure of the body 1 and the structure of the subject's body in the area of measurement.

The controller 3 analyzes the bio signals measured by the electrodes 2 to detect bio information according to a bio signal measurement request received from the mobile terminal 4 or inputted through the input unit 5 and transmits the detected bio information to the mobile terminal 4 using a radio signal.

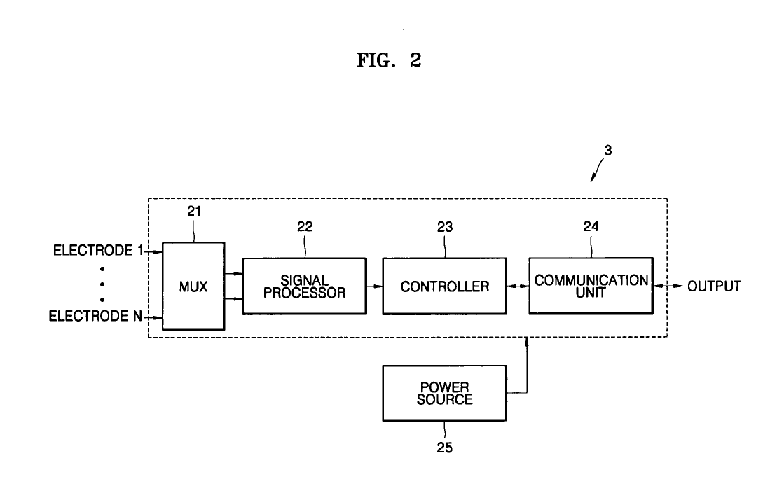

FIG. 2 is a block diagram of the controller of FIG. 1. Referring to FIG. 2, the controller 3 includes a multiplexer (MUX) 21, a signal processor 22, a bio information generator 23, a communication unit 24, and a power source 25.

The MUX 21 extracts two signals among the bio signals inputted from the plurality of electrodes 2. It is sufficient to be selected if the two signals have a potential difference between them. For example, two signals whose amplitudes are a first and second largest or two signals having the largest amplitude and the smallest amplitude, can be selected.

The signal processor 22 differential-amplifies the two signals selected by the MUX 21 and converts the differential-amplified signals to a digital signal.

The bio information generator 23 analyzes the bio signals outputted from the signal processor 22 to output the bio information. The bio information is bio information that corresponds to a portion of the human body to which the body 1 is attached. For example, in a case the body 1 is attached to a heart of the subject, a heart rate or an electrocardiogram can be used for the bio information. In another case, the body 1 is attached to an arm of the subject, an electromyogram can be used for the bio information.

The communication unit 24 outputs the bio information to the mobile terminal 4. The power source 25 supplies power required for the respective elements 21, 22, 23, and 24. A rechargeable secondary battery or a replaceable battery may also be used for the power source. The power source 25 can be turned on/off through the input unit 5 or by manipulation through the mobile terminal 4.

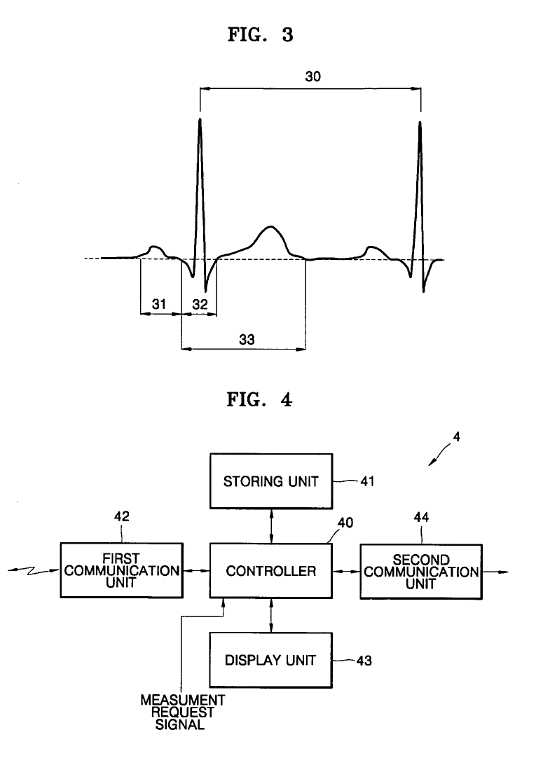

FIG. 3 illustrates two signals selected among the bio signals generated from the electrodes shown in FIG. 1. A waveform illustrates an electrocardiogram signal that can be measured when the body 1 is attached to the heart of the subject. A normal electrocardiogram signal includes an R-R time interval 30, a P-P time interval 31, a Q-T time interval 32, and a QRS time interval 33.

The R-R time interval 30 can be used for measuring a heart rate. Namely, the heart rate can be measured by counting a number of R peaks during a predetermined period of time. The P-P time interval 31 is an interval from a start of an atrial depolarization to a start of ventricular depolarization, i.e., a time from a P-wave to a start of a QRS complex. A normal range of the P-P time interval 31 is between 120 to 200 ms. The Q-T time interval 32 is a time from a start of the QRS complex to an end of a T-wave and a time of a sum of the ventricular depolarization and ventricular repolarization. A normal range of the Q-T time interval is below 440 ms. The QRS time interval 33 is a time during which the ventricular depolarization continues. A normal range of the QRS is below 120 ms.

The bio information generator 23 measures the respective time intervals that can be an important index of heart-related disease as shown in FIG. 3 to generate electrocardiogram data of the subject. The respective time intervals are determined by expressing the respective time intervals using a Gauss quadrature with respect to a potential difference signal outputted from the signal processor 22 and determining which shape of the expressed waveform is a first-order increase type, a first-order decrease type, a second-order increase type, a second-order decrease type, or a zero slope type. Such a method can be also applied in the same way to the electromyogram as in the electrocardiogram waveform.

FIG. 4 is a block diagram of an internal configuration of the mobile terminal 4 shown in FIG. 1. Referring to FIG. 4, the mobile terminal 4 includes a controller 40, a storing unit 41, a first communication unit 42, and a display unit 43. The mobile terminal 4 can also optimally have a second communication unit 44.

The first communication unit 42 communicates with the apparatus for measuring the bio signals of FIG. 1, transmits a measurement request signal from a subject delivered by the controller 40 to the apparatus for measuring the bio signals, and receives bio information from the apparatus for measuring the bio signals. The controller 40 stores the bio information in the storing unit 41 and displays the bio information on the display unit 43. The bio information stored in the storing unit 41 may be browsed later by the subject. The storing unit 41 can also store upper/lower permissible levels and/or a maximum upper permissible level and a minimum lower permissible level for the bio information. In that case, the controller 40 can inform the subject of an alarm through the display unit 43 if the bio information delivered through the first communication unit 42 gets out of the upper/lower permissible level. If the bio information gets out of the maximum upper permissible level and the minimum lower permissible level, the controller 40 can transmit an emergency signal informing that the subject is in an emergency state by connecting with an emergency center or a call center stored in the storing unit 41 through the second communication unit 44. If necessary, it is possible to allow a doctor to understand the state of the subject by transmitting current bio information of the subject and the bio information stored in the storing unit 41 together.

A programming environment having units and operations similar to that of the mobile terminal 4 shown in FIG. 4 is provided in a PDA and a personal computer (PC). For a mobile phone, a virtual machine-based environment such as a general virtual machine (GVM) is provided. Subsequently, functions of the controller 40 can be easily realized using the above-described programming environment. That is, the electrocardiogram measurement or the heart rate measurement function can be included as an additional function item in the mobile terminal 4.

According to the present invention, even if attached positions of the electrodes are changed by the subject's movement, a signal detection error can be prevented.

Since the electrodes can be attached in a natural and convenient way, the subject can live an everyday life without disturbance caused by the attachment of the electrodes. Further, the electrodes can make contact with the skin of the subject using a pack without electrolyte. It is also possible to analyze heart signals and connect with an emergency center in case of an emergency situation.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An apparatus for detecting bio signals, comprising:
a body filled with a predetermined packing material;
a plurality of electrodes positioned at an outermost portion of the body and configured to contact a subject's skin so as to measure bio signals from the subject; and
a controller positioned in an interior of the body and electrically coupled to the electrodes, that analyzes the bio signals measured by the electrodes to output bio information of the subject.

2. The apparatus of claim 1, wherein the body is flexible so as to be attached to an arbitrary portion on a body of the subject.

3. The apparatus of claim 1, wherein the packing material is one of liquid silicon, a sponge, and air.

4. The apparatus of clam 1, wherein the electrodes are electrically coupled to the controller through a conductive wire made of a flexible material.

5. The apparatus of claim 1, wherein the controller comprises:
a MUX (multiplexer) that selects two signals among the bio signals outputted from the plurality of electrodes;
a signal processor that differentially-amplifies the selected two signals into a differentially-amplified signal and converts the differentially-amplified signal into a digital signal;
a bio information generator that generates the bio information from the digital signal; and
a power source that supplies power to the MUX, the signal processor, and the bio information generator.

6. The apparatus of claim 1, further comprising a mobile terminal that receives the bio information from the controller and informs the subject of the bio information.

7. The apparatus of claim 6, wherein the mobile terminal outputs a bio signal measurement request signal to the controller and the controller measures the bio signal using the electrodes according to the bio signal measurement request signal.

8. A method for detecting bio signals, comprising:
attaching a body having a plurality of electrodes and filled with a predetermined packing material to a subject;
measuring the bio signals using the plurality of electrodes and selecting two bio signals among the bio signals; and
analyzing the two bio signals to generate bio information.

9. The method of claim 8, wherein the packing material comprises one of liquid silicon, a sponge, and air, so that the body is flexible.

10. The method of claim 8, wherein the two bio signals are two arbitrary bio signals having potential difference among the bio signals.

11. The method of claim 8, further comprising:
outputting the bio information to a mobile terminal; and
displaying, at the mobile terminal, the bio information.

12. The method of claim 11, further comprising the mobile terminal outputting an alarm if the bio information gets out of a predetermined threshold range.

13. The method of claim 12, further comprising if the bio information gets out of a maximum permissible threshold range, connecting the mobile terminal with a number or an address stored in the mobile terminal to transmit an emergency signal.
